(19) United States
(12) Patent Application Publication
(10) Pub. No.: US 2009/0054922 A1
(43) Pub. Date: Feb. 26, 2009

(54) Apparatus and Method for the Intravascular Control of Trauma

(76) Inventor: Harshal S. Broker, Dallas, TX (US)

Correspondence Address: CARSTENS & CAHOON, LLP, P O BOX 802334, DALLAS, TX, 75380, US

(21) Appl. No.: 11/844,166

(22) Filed: Aug. 23, 2007

(51) Int. Cl. A61M 25/10

(52) U.S. Cl. 606/194; 604/101.05; 604/509

(57) ABSTRACT

One aspect of the invention relates to a device for control of hemorrhage from major blood vessels in acute trauma. The device can be a self sizing expandable/collapsible device placed percutaneously across the site of injury and fitted with intravascular imaging to allow visualization of positioning without the need for x-ray equipment or an operating room. The expandable/collapsible device can be tapered to accommodate a large variance in vessel size and can be textured with treads to prevent movement in high flow vessels. The expandable/collapsible device can be placed at the patient's bedside, provides for vascular control during definitive repair, and can be removed after said repair.

### BACKGROUND OF THE INVENTION

1. Technical Field

The present invention generally relates to the treatment of blood vessel injury in trauma. Particularly the present invention provides an apparatus and method for the percutaneous placement of one or more intravascular devices for traumatic bleeding without the need for x-ray.

2. Description of Related Art

Traumatic bleeding from major blood vessels continues to be a leading cause of morbidity and mortality. The usual course in the present day is control of this hemorrhage with traditional surgical techniques. This includes invasive surgery for exposure of the injury, followed by proximal and distal control with traditional vascular clamps, and definitive repair of the injury. Without surgery, these conditions are uniformly fatal.

This invasive surgery approach leads to massive transfusion requirements and morbidity from the invasive nature of the surgery itself. Invasive surgery also requires transfer to a facility with personnel and equipment capable of performing such procedures. During this transfer, ongoing bleeding continues to occur. Even in successful procedures, massive transfusions raise the exposure to blood borne disease, multi-organ failure, infection, and costs.

Despite extraordinary advances in minimally invasive approaches to the treatment of blood vessels with stents and balloons, trauma continues to be managed in a traditional invasive fashion. This is due to the need for the expedient transfer of the patient to high level facility and the inability to use current endovascular devices due to the lack of time, training, and facility. Frequent deaths continue to occur due to a lack of vascular control during transfer and prior to traditional repair by a surgeon.

Prior art cases where patients who have been treated by minimally invasive means for trauma require a relatively stable patient, a high level facility with x-ray equipment, interventionalists, and interventional devices.

Also, as the population continues to age and the elderly patient more often becomes the trauma patient, there is a great need to develop minimally invasive procedures to limit the morbidity and mortality in these individuals from massive transfusions and invasive procedures.

The prior art describes the treatment of blood vessel injury by various methods with some involving the use of balloons and stents. For example, it is well known to interpose a balloon or stent within an injured segment of a blood vessel to exclude the injury.

Although there are a variety of stents to market which are available for such injuries, they are rarely used in trauma due to a multitude of disadvantages. There is often contamination in the trauma setting and thus permanent foreign bodies such as stents would portend infection. Stents require accurate sizing and do not span large variations in blood vessel size without the use of multiple components and detailed reconstructions and planning which is not possible in emergent situations.

Inflatable devices and balloons have also been described in the art but to date still fail to address the problem of the acute unstable patient in need of expedient hemorrhage control at the bedside. U.S. Pat. Nos. 4,183,102 by Guiset, 5,370,691 by Samson, 6,293,968 by Taheri, and 5,330,528 by Lazim, describe inflatable devices for supporting the vasculature, all of which fail to address the problem of an acute unstable patient in need of expedient hemorrhage control at bedside for the following reasons.

Guiset disclosed a plurality of hollow toroidal sleeves while Samson relates to a helically-wound polymeric tubing. Taheri disclosed an inflatable stent with a meshwork of intersecting conduits. Lazim disclosed an annular chamber with surrounding body with an outer chamber for flexible sleeve member. Each of these devices has various individual limitations. For example, they fail to provide a means for universal sizing when there is a large variance in blood vessel size, fail to provide a method for remaining in place under high flow velocities, fail to disclose a means for total flow occlusion during definitive repair, and/or fail to disclose a means to accurately define blood vessel anatomy so as to be accurately placed throughout the vasculature without x-ray and at the patient's bedside. This may explain the lack of their presence in contemporary trauma/vascular practice and the continued need for a minimally invasive apparatus and method in the emergent setting. Consequently, a need exists for an apparatus and method for treating vasculature trauma. Further, a need exists for an apparatus and method for providing a minimally invasive surgical procedure that can be used in an emergency situation to control traumatic bleeding.

### SUMMARY OF THE INVENTION

The present invention is directed towards an apparatus and method for the intravascular control of traumatic bleeding. In one embodiment, a catheter comprising a flexible rigid tube having an intravascular imaging device for providing real-time imaging of a vessel or organ. One or more expandable/collapsible devices are attached to the catheter.

In one aspect, the invention provides an apparatus and method for intravascular control of bleeding without the need for an x-ray and that can be performed at the bedside of the individual or even in the field including, but not limited to, military and automobile accident settings. In one aspect, the invention provides a way to continue vascular control during repair of the vessel. These and other advantages of the present invention will become evident upon a review of the following description. It will be understood that the description, which is to be read with reference to the drawings, is given by way of example only and not by way of limitation.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 illustrates a cross-sectional side view of the vascular catheter in accordance with one embodiment of the present invention;

FIG. 2 illustrates a cross-sectional end view of the vascular catheter in accordance with one embodiment of the present invention;

FIG. 3 illustrates a perspective side view of the vascular catheter in the expanded position in accordance with one embodiment of the present invention; and

FIG. 4 illustrates the catheter utilized in a multitude of traumatic injuries in accordance with various embodiments of the present invention.

### REFERENCE NUMERALS

1—wire port
2—outer catheter assembly
3—distal balloon injection port
4—middle balloon injection port
5—proximal balloon injection port
6—intravascular imaging catheter
7—external imaging element
8—allocated portion of the catheter tubing for proximal balloon
9—allocated portion of the catheter tubing for middle balloon
10—allocated portion of the catheter tubing for distal balloon
11—allocated portion of the catheter tubing for the intravascular imaging catheter
12—flow passage way
13—flow passage way for middle balloon
14—flow passage way for distal balloon
15—distal balloon
16—middle balloon
17—proximal balloon
18—end hole for wire
19—echogenic etch marks on the device at locations near the tip
20—echogenic etch marks on the device at locations between balloon components
21—intravascular imaging element at catheter tip
22—intravascular imaging element between balloon components
23—proximal palpable radio-dense markers
24—wire port channel
25—expanded proximal balloon
26—expanded middle balloon
27—expanded distal balloon
28—proximal palpable radio-dense markers
29—proximal palpable radio-dense markers
30—distal palpable radio-dense markers
31—distal palpable radio-dense markers
32—distal palpable radio-dense markers
33—tread markings
34—tread markings
35—tread markings
36—distal end of middle balloon
37—proximal end of middle balloon
38—middle balloon thickness
39—central passageway diameter
40—retro-hepatic Inferior Vena Cava (IVC) injury
41—non tapered middle balloon
42—distal balloon
43—proximal balloon
44—intravascular imaging element
45—central flow channel of the tubular balloon
46—bleeding from the internal iliac artery
47—intravascular imaging element
48—middle balloon
49—common iliac artery
50—external iliac artery
51—central flow channel
52—subclavian artery injury
53—intravascular imaging element
54—thoracic aorta
55—distal balloon
56—middle balloon
57—central flow channel
58—proximal balloon
59—a position in the thoracic aorta
60—intravascular imaging element
61—aortic occlusion balloon
62—middle balloon
63—level of the renal artery
64—superior mesenteric artery
65—celiac artery
66—tread markings
67—renal injury
68—intravascular imaging element
69—tubular balloon
70—injury to the spleen
71—aortic injury
72—intravascular imaging element
73—aorta
74—common iliac artery
75—tapered tubular balloon
76—central flow channel

### DETAILED DESCRIPTION

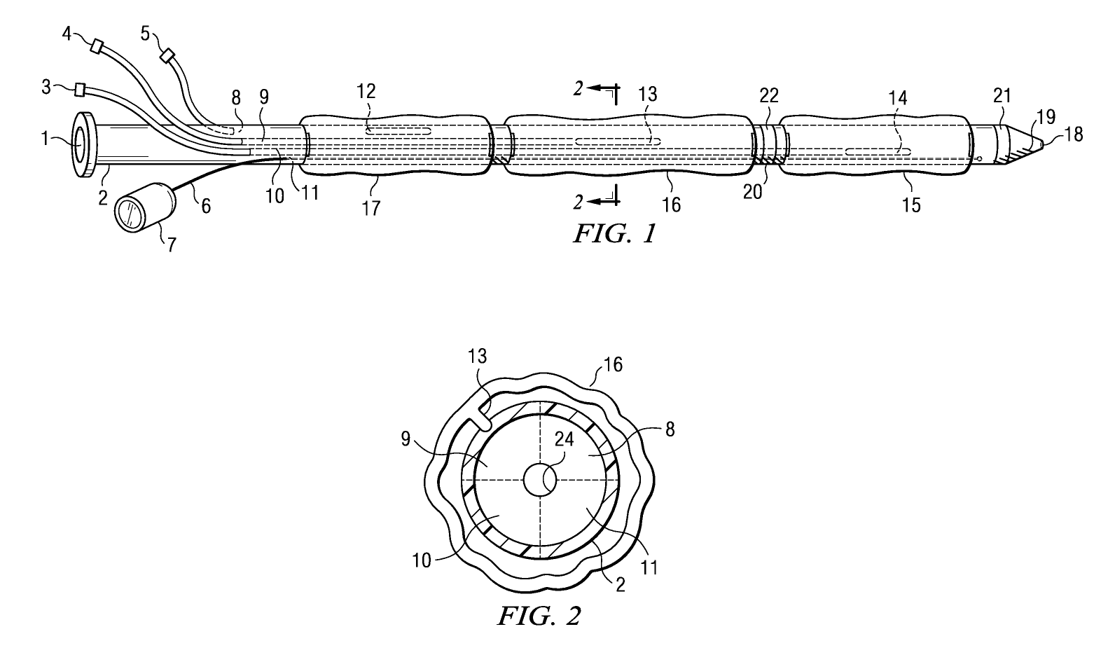

The present invention is an apparatus for the intravascular control of trauma or injury to blood vessels. FIG. 1 is a cross-sectional side view of the vascular catheter in the deflated position in accordance with one embodiment of the present invention. The wire port 1 accommodates a guide wire for use as in Seldinger technique but may also be used as an injection port for angiography once the patient is in an operating theater. The end hole for wire port 18 is located at the tip of the device. In one embodiment, one or more balloons comprise a hydrophilic outer layer to permit the catheter to slide into the vessel without the use of a guidewire.

Echogenic etch marks 20 can be placed on the device at locations near the tip 19, in an area 22 between expandable/collapsible devices or other desired locations. These echogenic etch marks confer acoustic properties to make said device more visible during traditional ultrasound techniques as described by U.S. Pat. No. 4,401,124 by Guess.

An intravascular imaging element may be placed in one or more locations such as the catheter tip 21 or in an area 22 between balloon components. Although the echogenic etch marks 20 are shown in an area 22 between the distal balloon 15 and the proximal balloon 17, those skilled in the art will understand such marks can be placed elsewhere including between other balloons, such as between the proximal balloon 17 and middle balloon 16 or other places on the catheter. The present application also incorporates an intravascular imaging element such as an intravascular ultrasound catheter (“IVUC”) which is known in the art. IVUCs are able to provide real-time imaging of the internal anatomy of blood vessels and other passageways without the need for cumbersome x-ray equipment.

Examples of these devices that have recently been disclosed include U.S. Pat. Nos. 5,749,848 by Jang, 4,917,097 by Proudian, 6,440,077 by Jung, 7,179,270 by Makower, and 6,780,157 by Stephens. These patents referenced herein provide general discussion for the use of IVUC in balloon and subintimal angioplasty, Inferior Vena Cava Filter placement, and other general intravascular imaging techniques and are hereby incorporated by reference. To the extent there is any conflict in definitions or terms, this disclosure controls.

Such catheters are commonly within a plastic sheath having a circumferential wall enclosing and protecting the internal circuitry of the IVUC. It is not the objective of the present invention to further develop or redesign the construction of such catheters, but rather seek to employ an intravascular imaging element such as an intravascular ultrasound catheter in a novel device. Prior art uses of an IVUC are almost exclusively for diagnostic procedures. An example of one prior art use of the IVUC is to ascertain the level of blockage in a blood vessel. Once the level of blockage has been ascertained, the IVUC is removed and any further procedures in the placement of a stent is carried out under X-Ray. The prior art herein mentioned fails to provide for the ability to utilize an IVUC device in an emergency situation to control traumatic bleeding with balloon/stent occlusion method because such prior art catheters require x-ray equipment to determine landmarks for placement of balloons, requiring removal of the IVUC catheter, and then placement of the balloon/stent without real-time IVUC imaging, and therefore an educated guess as to location.

As used herein, the term “expandable/collapsible device” is used interchangeably with the term “balloon” both terms refer to a device that can reversibly actuated between an expanded position and a collapsed position. The expandable/collapsible device can be fluid actuated. For example, a balloon can be expanded via catheter tubing in fluid communication with a balloon and an injection port. In an alternative embodiment, the expandable collapsible device can be mechanically actuated. Any suitable expandable/collapsible device can be used in accordance with the scope of the claimed invention including, but not limited to, any suitable balloon including a total occlusion balloon and a tubular balloon, a self-expanding stent, a balloon expandable stent, and retrievable stents. In one embodiment, the expandable/collapsible device is self sizing meaning that it expands to conform to the inside cross-section of the passageway such as a vessel. Consequently, in one embodiment, the expandable/collapsible device, especially if tapered, can accommodate a variance in vessel size.

The outer catheter assembly 2 comprises a flexible, rigid tube so as to support the device against high flow rates pushing against the occlusion balloons. In one embodiment, an expandable/collapsible device comprises a tubular or total occlusion balloon and catheter tubing disposed within a rigid tube 2.

In one embodiment, the Distal Balloon Injection port 3 allows for inflation and deflation of a distal balloon 15 within the allocated portion of the catheter tubing 10 with saline or contrast material via the flow passage way 14 for total blood flow occlusion. This balloon and other components herein may be optional so as to customize the device for the appropriate clinical situation.

In on embodiment, the Middle Balloon Injection port 4 allows for inflation and deflation of a middle balloon 16 within the allocated portion of the catheter tubing 9 with saline or contrast material via the flow passage way 13. This flow passage way 13 is located in the middle of the balloon so as to inflate or expand the balloon in the center and then out proximally and distally so as to reduce drag that may occur during high flow states.

In one embodiment, the proximal balloon injection port 5 allows for inflation and deflation of the proximal balloon 17 within the allocated portion of the catheter tubing 8 with saline or contrast material via the flow passage way 12 to control back bleeding when expanded. The specific balloon combination can be easily adjusted to customize the catheter assembly as needed.

The allocated portion 11 of the catheter tubing for the intravascular imaging catheter is illustrated. The intravascular imaging catheter 6 terminates at an external imaging element 7 that may connect to a computer processor and imaging screen. The imaging element 7 may also be a wireless transmitter to said processor in an effort to improve efficacy in the emergency room.

FIG. 2 illustrates a cross-sectional end view of the vascular catheter in accordance with one embodiment of the present invention. The outer catheter assembly portion of the device 2 may be of any suitable synthetic plastic-like material to house the various device components in a rigid fashion. The wire port channel 24 is seen in cross-sectional view. The allocated portions of catheter tubing for the distal balloon 10, middle balloon 9, proximal balloon 8, and the intravascular imaging catheter 11 containing wire and circuitry are seen in cross-section. In this cross section, the middle balloon 16 is seen encompassing the outer catheter assembly 2 and the flow passage way 13 to the middle balloon 16 is visualized. Although in this depiction, the area that the various components occupy is equal, this of course may be modified so as to give larger components more space and smaller components less space within the catheter.

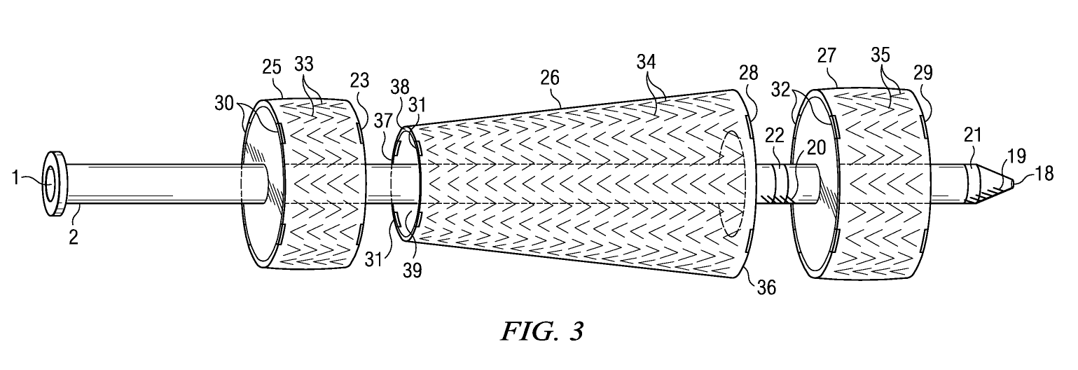

FIG. 3 illustrates a perspective side view of the vascular catheter in the expanded position in accordance with one embodiment of the present invention. The wire port 1, the catheter assembly 2, end hole for the wire port 18, echogenic etch marks 19 20, and intravascular imaging elements 21 22 are once again depicted. The distal balloon 27, middle balloon 26, and proximal balloon 25 are seen in their expanded states. During device manufacturing, one or more of these balloons may be removed to accommodate different clinical situations. The middle balloon 26 may be of a tubular shape to oppose the vessel wall yet maintain vessel patency via a center flow channel. In one embodiment, the middle balloon 26 is tapered as depicted with the distal end of the middle balloon 36 having a larger diameter than the proximal end 37. Such tapering can help the balloon 26 advantageously accommodate a large variance in blood vessel size that is often seen. The middle balloon 26 may also be manufactured with equal diameters on its proximal end 37 and distal end 36. Additionally, FIG. 3 illustrates proximal 23, 28, 29, and distal palpable radio dense markers 30, 31, 32. These markers label the proximal and distal extent of the balloons. During definitive repair, it is often important to know where the balloons begin and end by feel or by fluoroscopy. These markers may be made of a gold alloy or other palpable radio dense material.

One of the main problems with using balloon occlusion in major vessels is the tendency for the balloon to migrate in high flow vessels. This migration leads to loss of vascular control and bleeding. Consequently, tread markings 33, 34, 35 can be provided on the balloon surfaces. The pattern of tread markings 33, 34, 35 depicted in the Figure are shown only as an example of the tread markings and in no way limits the possible configurations to maximize traction for the balloon against the vessel wall. The treads may be channels created within the balloon wall or as protrusions affixed to the balloon with adhesive or suture material. The protrusions may be made of materials such as latex, PTFE, or other plastics.

The thickness 38 of the middle balloon 26 in the expanded state can be adjusted to increase or decrease the diameter of the middle balloon 26 central passageway 39, which is defined by the inner diameter of the balloon 26. Such adjustment can advantageously adjust flow. For example, the diameter of the middle balloon central passageway 39 can be increased to provide maximal flow or decreased to reduce flow as specific clinical situations may dictate.

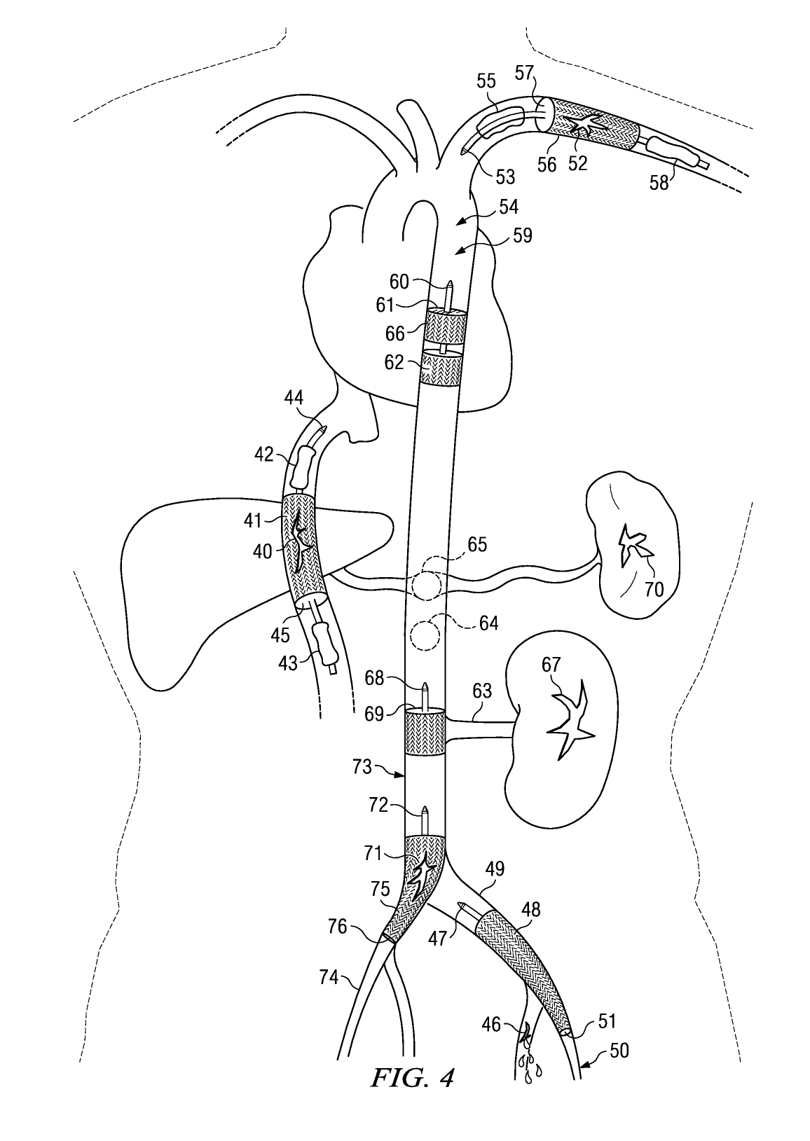

FIG. 4 illustrates the catheter utilized in a multitude of traumatic injuries in accordance with various embodiments of the present invention as described more specifically in the six Examples set forth below. The injury itself may be identified with the intravascular imaging device and projected to a video screen (not shown) via the external imaging element 7 shown in FIG. 1. A cross-sectional image of the blood vessel is generated from the ultrasonic imaging element to provide real-time imaging of catheter/balloon location as it navigates a blood vessel. As the catheter is guided through a blood vessel, an attending physician is able to determine the catheter location based on branched vessels, changes in vessel diameter, and surrounding organs, which can be ascertained based on the cross sectional displayed on a video screen. As most all humans have the same blood vessel configuration, the real-time imaging provides the physician a precise awareness of the catheter location as the catheter navigates any body passageway, including blood vessels and organs.

### EXAMPLE 1

A retro-hepatic Inferior Vena Cava (IVC) injury 40 is seen. Mortality from this injury is commonly above 90%. Prior art such as U.S. Pat. No. 6,325,776 by Anderson describe the traditional invasive approach requiring a exposure via a large incision in the chest (thoracotomy or sternotomy) and laparotomy prior to control of bleeding being obtained. With the present device, minimally invasive percutaneous control may be obtained. The apparatus similar to that depicted in FIG. 3 is used. However, instead of using a tapered middle balloon 26, as depicted in FIG. 3, a vascular catheter having a non-tapered middle balloon 41 is used. The vascular catheter is placed into the right or left femoral vein and advanced with ultrasound guidance from the intravascular imaging element 44 to a point past the injury 40. A tubular, non-tapered middle balloon 41 is inflated or expanded to prevent blood loss through the injury 40. Consequently, immediate control of the traumatic bleeding is obtained while blood is still able to flow in the central flow channel 45 of the tubular balloon 41. For definitive repair, the distal total occlusion device 42 and proximal total occlusion balloon 43 may be expanded or inflated and the middle balloon 41 deflated or collapsed while the injury is repaired or surgical control is obtained.

### EXAMPLE 2

Pelvic fractures are a very common type of trauma after motor vehicle accidents. They often lead to bleeding from the internal iliac artery 46 or its braches. Definitive treatment is usually coil emoblization of the internal iliac artery 46 but this takes a dedicated angiographic suite with a physician interventionalist. During the transfer to a facility with these capabilities, blood is lost and hematoma formation can cause injury to other organs and nerves, massive transfusion, infection, other morbidity, and often death. Percutaneous control may be obtained with the present device by using the intravascular imaging element 47 to determine the location of the internal iliac artery 46 and the middle tubular balloon 48 may be inflated or expanded to exclude the injury until definitive repair may be performed, or possibly tamponade the bleeding with no further treatment needed. The middle tubular balloon 48 is tapered to allow for the large change in diameter from the common iliac artery 49 to the external iliac artery 50. The central flow channel 51 permits flow to the left leg while transfer or repair is performed.

### EXAMPLE 3

A subclavian artery injury 52 is illustrated. The vascular catheter may be placed via the left axillary or brachial artery. The intravascular imaging element 53 is advanced past the injury 52. If the intravascular imaging element 53 goes in too far and lands in the thoracic aorta 54, the device is simply pulled back in to the correct location. The middle balloon 56 is inflated or expanded and control of bleeding is obtained. The central flow channel 57 allows blood to flow into the left arm during transfer to definitive care. At the time of definitive repair, the middle balloon 56 is deflated or collapsed and the distal balloon 55 and proximal balloon 58 are inflated or expanded to obtain proximal and distal control.

### EXAMPLE 4

Although many occlusion balloons exist for the aorta, trauma surgeons and emergency physicians still routinely perform an aortic cross clamp with an emergency room resuscitative thoracotomy (“ER Thoracotomy”) when a patient is in extremis and bleeding to death. This procedure is morbid and time consuming with a mortality approaching 99% for abdominal trauma and 85-98% for thoracic trauma. It also carries the risk of transmission of blood borne disease as many sharp objects are involved in this emergency procedure performed in the emergency room crowded with personnel. The reason this procedure is still performed despite aortic occlusion balloon being available include the inability to determine position of the balloon, balloon migration after deployment, and time consumption involved in obtaining x-rays. In FIG. 4 an aortic occlusion balloon is in place in the thoracic aorta 61. The device was advanced from the femoral artery with the intravascular imaging element 60 used as a guide to advance it above the level of the renal artery 63, the superior mesenteric artery 64, the celiac artery 65, and to a position in the thoracic aorta 59. At this point the distal occlusion balloon 61 with the tread markings 66 is expanded. If further stabilization is needed, the middle balloon 62 may also be expanded. At this point, the equivalent of an aortic cross clamp is achieved and definitive care may proceed. There would be no need for a resuscitative thoracotomy and aortic cross clamp.

### EXAMPLE 5

Renal trauma often leads to nephrectomy due to extensive bleeding and morbidity involved in dissecting the kidney in the setting of active bleeding. Kidney preservation would be more likely if control of bleeding were obtained prior to definitive repair. A renal injury is depicted 67. The device is advanced using the intravascular imaging element 68 to a point past the renal artery 63, but below the level of the superior mesenteric artery 64. At this point the tubular balloon 69 (notice absence of proximal and distal occlusion balloons in this version of the device) is expanded and control of the renal artery 63 is obtained. A similar concept may be utilized for injury to the spleen 70, and coverage of the celiac artery 65 with said tubular balloon 69.

### EXAMPLE 6

An aortic injury is seen 71. The device is place up the right femoral artery with the intravascular imaging element 72 guided past the injury. The tapered tubular balloon 75 is expanded and the bleeding is controlled. The tapered tubular balloon 75 allows for the large change in diameter from the Aorta 73 to the common iliac artery 74 in an aorto-uniiliac configuration. The central flow channel 76 permits flow to the right leg while transfer or repair is performed. This same concept may be applied if the aortic injury 71 is actually a ruptured aortic aneurysm. The intravascular imaging element 72 guides the device past the aneurysm to a level below the renal arteries 63 and a tapered balloon 75 may be expanded from a point at the renal arteries 63 all the way down to a point in the common iliac artery 74. This would exclude the bleeding aortic injury 71 caused by rupture of an aneurysm (not depicted).

While this invention has been particularly shown and described the preference to the preferred embodiment, it will be understood by those skilled in the art that changes in form and detail may be made therein without departing from the spirit and scope of the invention.

What is claimed is:

1. A catheter for an emergency medic, said catheter comprising:
a flexible, rigid tube, said rigid tube comprising:
an intravascular imaging device for providing real-time imaging of a passageway;
at least one expandable/collapsible device in a collapsed state wherein when said catheter is inserted in a percutaneous fashion into said passageway, and said catheter is guided based on said real-time imaging to an area adjacent to an area of injury, and said expandable/collapsible device is expanded from the collapsed state into an expandable state.

2. The catheter of claim 1 further comprising three balloon assemblies including:
a proximal expandable/collapsible device having a proximal total occlusion device;
a expandable/collapsible device having a middle tubular device; and
a expandable/collapsible device having a distal total occlusion device, such that definitive repair is possible with inflation of said proximal total occlusion device and said distal total occlusion device.

3. The catheter of claim 1 wherein said expandable/collapsible device comprises a total occlusion device to encompass the entirety of the blood vessel lumen.

4. The catheter of claim 1 wherein said expandable/collapsible device comprises a tubular device to provide a center flow channel to provide blood flow while maintaining control of bleeding across an injury.

5. The catheter of claim 1 wherein said expandable/collapsible device comprises a tapered device.

6. The catheter of claim 1 wherein said expandable/collapsible device further comprises tread markings so as to provide traction and stability of said device in blood vessels.

7. The catheter of claim 1 wherein said rigid tube further comprises:
a guidewire endhole;
a wire port at a proximal end; and
a wire port channel disposed between said guidewire endhole and said wire port.

8. The catheter of claim 1 further comprising echogenic etch marks.

9. The catheter of claim 1 wherein said device further comprises tread markings.

10. The catheter of claim 1 Comprising a total occlusion device and tubular device.

11. A method for the intravascular control of hemorrhage from major blood vessels in acute trauma comprising the sequential steps of:
a) providing a catheter having at least one intravascular imaging device for providing real-time imaging of a body passageway and at least one expandable/collapsible device;
b) guiding said catheter into a blood vessel in open or percutaneous fashion to an area adjacent said hemorrhage; using said real-time imaging as a guide for location of said device without the use of an x-ray, and
c) expanding said device.

12. The method of claim 11 wherein said balloon comprises a total occlusion device said guiding at step b) comprises inserting said total occlusion device past said area of injury.

13. The method of claim 11 wherein said balloon comprises a total occlusion device to block blood flow.

14. The method of claim 11 wherein said balloon comprises a tubular device to permit blood flow.

15. The method of claim 11 wherein said catheter at step a) comprises:
a proximal expandable/collapsible device having a proximal total occlusion device;
a middle expandable/collapsible device having a middle tubular device; and
a distal expandable/collapsible device having a distal total occlusion device,
wherein said guiding a step b) further comprises placing said middle tubular balloon across the injury, and wherein said expanding at step c) comprises expanding said distal total occlusion device, and expanding said tubular device.

16. The method of claim 15 wherein said middle device is tapered

17. The method of claim 15 further comprising the step of collapsing said distal total occlusion device after step c).
